(19) United States
(12) Patent Application Publication
(10) Pub. No.: US 2009/0075318 A1
(43) Pub. Date: Mar. 19, 2009

(54) USING PHOTO-RESPONSIVE SURFACTANTS TO REVERSIBLY CONTROL PROTEIN AGGREGATION WITH LIGHT ILLUMINATION

(75) Inventors: Charles Ted Lee, JR., Pasadena, CA (US); Andrea C. Hamill, Falls Church, VA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/205,515

(22) Filed: Sep. 5, 2008

Related U.S. Application Data
(60) Provisional application No. 60/970,206, filed on Sep. 5, 2007.

(51) Int. Cl. C12Q 1/37; G01N 33/00; C12N 9/76; C07K 1/00

(52) U.S. Cl. 435/23; 436/86; 530/402; 435/213

(57) ABSTRACT

The present invention relates to methods of inducing protein folding using light illumination. More specifically the invention relates to shape-reconstruction analysis applied to small angle neutron scattering (SANS) data that is used to determine the structure of partially-folded proteins in non-native conformations and supramolecular complexes undergoing self- or hetero-association in solution as a result of partial unfolding with a photoresponsive surfactant.

### STATEMENT REGARDING FEDERALLY SPONSORED RESEARCH OR DEVELOPMENT

Support from National Science Foundation (CBET-0554115) is acknowledged.

### FIELD OF THE INVENTION

The present invention relates to the use of photo-responsive surfactants to reversibly control protein aggregation with light illumination.

### BACKGROUND OF THE INVENTION

Proteins interact with a variety of molecules during the course of activity, ranging from small ions and ligands to other proteins through either heterogeneous or homogenous association. Indeed, the dynamic and multifarious response of proteins to these interactions is utilized to stimulate or regulate virtually every biological process. In some cases, however, protein interactions can result in unwanted or deleterious effects, such as protein-protein associations leading to amyloid fibril formation. The most well-known example of this process involves the aggregation of the amyloid-beta (Aβ) peptide fragments Aβ40 and Aβ42 implicated in Alzheimer's disease, although amyloid fibrils have been observed in an array of proteins largely independent of the native secondary structure, (1) including ribonuclease A, (2) an SH3 domain, lysozyme, (3) insulin, (4) and α-chymotrypsin. (5) This process is generally believed to result from the formation of unstable slightly-unfolded conformations, leading to a cascading aggregation process from monomers to oligomers (unstructured aggregates of typically multiples of six molecules in the case of Aβ42(6)) to protofibrils (structured aggregates exhibiting β-sheet structure) to protofilaments (elongated aggregates ˜2-5 nm in diameter) to fibrils (2-6 entwined protofilaments). (1) Perhaps most importantly, the prefibrillar intermediates (i.e., oligomers and protofibrils), which can induce cognitive impairment, have become increasingly viewed as the primary pathogenic species. (1, 7)

To date, however, the solution structure of these important intermediate species remains unknown as the two preferred methods to determine protein structure, namely X-ray crystallography and solution NMR, are generally limited to the study of native proteins in the solid state or relatively small protein assemblies, respectively, since protein crystallization is often supplanted by unwanted aggregation and crystal-packing constraints largely dominate protein orientations in multi-molecular complexes. Thus, the development of novel structural characterization methods capable of examining partially-folded proteins in non-native conformations and supramolecular complexes undergoing self- or hetero-association is highly desired. For example, through the use of small-angle neutron scattering (SANS), the low-resolution in vitro structures of Aβ40 protofibrils were found to by cylindrical with cross-sectional radii of 24 Å and 110 Å long, (8) while through AFM and TEM measurements a variety of protofibril arrangements have been observed, including twisted chains 2-5 nm in diameter. However, without higher-resolution conformations, and due to potential influences of surface interactions, the precise nature of protofibrils conformation in solution remains unknown. As a result, to properly investigate intermediate conformations in an amyloid protein necessitates two complementary approaches: (1) a means to induce changes in protein folding and, hence, association in a controlled and preferably reversible manner, and (2) a method to determine the conformation of non-native and associated proteins at relatively high resolution.

Recently, the inventors have shown that light illumination can be used to induce photoreversible changes in both the secondary (9) and tertiary (10, 11) structure of proteins. This method utilizes the interaction of proteins with photosensitive “azoTAB” surfactants containing an azobenzene group that undergoes a trans (relatively hydrophobic) to cis (relatively hydrophilic) photoisomerization upon exposure to visible (434 nm) or LV (350 nm) light illumination, respectively. Hence, light can be used to reversibly bind the surfactant to the hydrophobic domains of proteins, leading to photocontrol of protein folding. Furthermore, the inventors have applied small-angle neutron scattering (SANS) to study the in vitro structure of the non-native protein conformations that form in response to photosurfactant and light. Small-angle neutron and X-ray scattering have been used for several decades to investigate the structure of soluble proteins in solution (12-15) and membrane proteins in surfactant assemblies (16, 17). The obtained structures have typically been low-resolution, however, a consequence of modeling proteins with a single dimension (radius of gyration) or as ellipsoids (axial radii). These procedures, although convenient, belie the wealth of structural information contained within the measured scattering intensity. From the range of momentum vectors Q=4πλ⁻¹ sin(θ/2), where λ is the neutron wavelength (6 Å) and θ is the scattering angle, in a typical SANS experiment (Q=0.005-0.5 Å⁻¹), it can be seen that the data span length scales (L=2π/Q) ranging from 12.5-1250 Å, ideal for protein conformational studies. Indeed, application of shape-reconstruction techniques such as the ab initio methods of GASBOR (14) and GA_STRUCT (15) reveals a high degree of similarity between the native structure in solution (SANS) and in the solid state (X-ray crystallography), a seemingly general property of soluble proteins. (12)

### SUMMARY OF THE INVENTION

In the present invention, the ability to photo-initiate changes in protein quaternary structure through photoreversible control of α-chymotrypsin self-association is demonstrated. Native α-chymotrypsin is well-known to self-associate through either a monomer-dimer (pH 3) or monomer-hexamer (pH 7) equilibrium, while the addition of trifluoroethanol, a solvent known to induce partially-folded structures (18, 19) has been reported to result in α-chymotrypsin amyloid-fibril formation. (5) Mixing α-chymotrypsin with the photoresponsive azoTAB surfactant is found to result in partial unfolding of the protein, giving rise to changes in both the degree and type of self-association. Shape-reconstruction analysis applied to SANS data allows determination of the in vitro conformation of α-chymotrypsin oligomers. In the presence of azoTAB under visible light, native oligomers (dimer or compact hexamers) are converted to expanded corkscrew-like hexamers, while upon UV-light illumination the hexamers laterally aggregate, wrapping around each other to form dodecamers with twisted conformations. FT-IR measurements of the protein secondary structure reveal that dodecamer formation is accompanied by hydrogen-bond stabilized intermolecular β sheets, commonly observed in amyloid fibrils. TEM measurements following incubation further confirm to formation fibrillar structures, while, photo-reversibility of the hexamer-to-dodecamer association process is studied with small-angle X-ray scattering (SAXS) measurements. Together, these results provide what is believed to be the first direct observation of the mechanism of formation of the key intermediates in an amyloid-forming protein, which should provide unique insight into the amyloidosis disease pathway.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### DESCRIPTION OF THE FIGURES

FIG. 1. SANS data of α-chymotrypsin/azoTAB solutions as a function of surfactant concentration and light illumination at (a) pH 3.0, 11.6 mg/mL protein, and (b) pH 7.2, 11.4 mg/mL protein.

FIG. 2. SANS data of pure α-chymotrypsin solutions at (a) pH 3 and (c) pH 7: raw data (), scaled PBD (2CHA) (), and raw data minus the scaled PDB (◯). Insets show Guinier analysis of the raw data. Also shown are pair distance distribution functions at (b) pH 3.0 and (d) pH 7: raw data () and oligomer-only data ( . . . ). For comparison the raw data minus the oligomer data ( . . . ) and 2CHA [symbol] are displayed.

FIG. 3. Shape-reconstructions of the oligomer-only SANS data for pure α-chymotrypsin at (a) pH 3 and (b) pH 7 (in blue) compared to the X-ray crystallographic structures of the α-chymotrypsin dimer (PDB code 6CHA) and chymotripsinogen-A dimer (PDB code 2CGA) at pH 3. The insert shows a hypothetical hexamer built from three 2CGA subunits (alternating monomers shown in blue and green) along with the consensus envelope and worst fit (shown in red).

FIG. 4. Guinier analysis of the raw SANS data. (a) pH 3: pure α-Ch (), 1.59 mM azoTAB visible (♦) and UV (⋄), 4.23 mM visible (▪) and UV (□), 6.70 mM visible (▴) and UV (Δ). (b) pH 7: pure α-Ch (), 1.03 mM visible (♦) and UV (⋄), 4.03 mM visible (▪) and UV (□), 9.92 mM visible (▴) and UV (Δ).

FIG. 5. Pair distance distribution functions of the oligomer-only data scaled by the respective oligomer weight fractions. (a) pH 3: 1.59 mM azoTAB visible (♦) and UV (⋄), 4.23 mM visible (▪) and UV (□), and 6.70 mM visible (▴) and UV (Δ). (b) pH 7: pure α-Ch (), 1.03 mM visible (♦) and UV (⋄), 4.03 mM visible (▪) and UV (□), and 9.92 mM visible (▴) and UV (Δ). Also shown are the SANS scattering curves of the oligomer-only data. (c) pH 3: 1.59 mM azoTAB visible (♦) and V (⋄), 4.23 mM visible (▪) and UV (□), and 6.70 mM visible (▴) and UV (Δ). (d) pH 7: pure α-Ch (), 1.03 mM visible (♦) and UV (⋄), 4.03 mM visible (▪) and LUV (□), and 9.92 mM visible (▴) and LUV (Δ).

FIG. 6. Shape-reconstructions of the oligomer-only SANS data at (a) pH 3 and (c) pH 7 as a function of azoTAB concentration and light illumination. Inserts show four views of the shape reconstructions rotated at 90°, along with low-resolution globular models designed to mimic the twisted conformations detected in the structures.

FIG. 7: (a) FT-IR absorbance spectra for pure α-Ch (black) and mixtures of α-Ch with 9.04 mM azoTAB under visible light (red) and UV light (blue). (b) FT-IR difference spectra (UV—visible) demonstrating the effect of light illumination. Also shown are Congo red fluorescence (c) and apple green birefringence (d) obtained under cross polarizers, as well as TEM images of a fresh solution (e-f) (pH3, [azoTAB]=4.95 mM) and an original SANS solution (g) (pH3, [azoTAB]=4.23 mM) after an elapsed time of approximately one year.

FIG. 8: SAXS data of chymotrypsinogen-A/azoTAB solutions as a function of surfactant concentration and light illumination at pH 2.7 and 10.0 mg/mL protein under (a) visible light and (b) UV light. Pure α-Ch (), 2.5 mM azoTAB [symbol] 5 mM [symbol] 10 mM [symbol] 14 mM (♦), 19 mM (▪), and 24 mM (▴). Also shown are the effects of re-illuminated with visible light on UV-adapted samples at various exposure times: 19 mM, 1 hr (□), 14 mM, 1 hr [symbol] , 10 mM, 1 hr (⊕) and 2 hrs [symbol] Insets show Guinier fits.

### DETAILED DESCRIPTION OF THE INVENTION

Shape-reconstruction analysis applied to small angle neutron scattering (SANS) data was used to determine the in vitro conformations of α-chymotrypsin oligomers that form as a result of partial unfolding with a photoresponsive surfactant. In the presence of the photo-active surfactant under visible light, the native oligomers (dimers or compact hexamers) rearrange into expanded corkscrew-like hexamers. Converting the surfactant to the photo-passive form with UV-light illumination causes the hexamers to laterally aggregate and intertwine into dodecamers with elongated, twisted conformations containing cross-sectional dimensions similar to amyloid protofilaments. Secondary-structure measurements with FT-IR indicate that this photo-induced hexamer-to-dodecamer association occurs through intermolecular β sheets stabilized with hydrogen bonds, similar to amyloid formation. SANS is ideally suited to the study of these associated intermediates, providing direct observation of the mechanism of oligomeric formation in an amyloid-forming protein. Combined with photoreversible hexamer-to-dodecamer associations in the presence of the photoresponsive surfactant, the present invention should provide insight into the amyloidosis disease pathway, as well as disease treatment strategies.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Experimental Methods

An azobenzene-trimethylammonium bromide surfactant (azoTAB) of the form

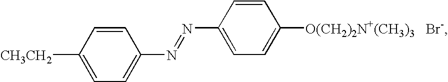

similar to surfactants used in previous studies, (10, 11, 20, 21) was synthesized according to published procedures. (22, 23) When illuminated with 350-nm UV light, the surfactant undergoes a photoisomerization predominantly to the cis form (90/10 trans/cis), which can be rapidly reversed upon exposure to visible light (434 nm, 75/25 trans/cis) or in the dark in about 24 hours (˜100% trans isomer). (20) For the SAXS and FT-IR measurements, conversion to the UV-light form was achieved with the 365-nm line from a 200 W mercury arc lamp (Oriel, model 6283), isolated with the combination of a 320-nm band-pass filter (Oriel, model 59800) and an IR filter (Oriel, model number 59060). A 400-nm long-pass filter (Oriel, model 59472) was used to convert back to the visible-light form. In the SANS experiments, the solutions were exposed to an 84 W long-wave UV lamp (365 nm, Spectroline, model XX-15A) for at least 30 min prior to sample collection to convert to the UV-light form, and were continuously exposed to the same UV light throughout the data collection.

Type II, essentially salt-free α-chymotrypsin from bovine pancreas (Sigma, cat. # C-4129, lot # 105K7670), 5× crystallized chymotrypsinogen-A from bovine pancreas (Worthington, cat. # LS005630), and phosphate buffer (Sigma, cat. # P-3288, pH 7.2, 8.3 mM) were used as received. All other chemicals were obtained from Aldrich in the highest purity. For the experiments preformed at pH 3, HCl (37%) was added to the pH 7.2 buffer as needed.

Small-angle neutron scattering experiments were performed on the 30-m NG3 SANS instruments at NIST. (24) A neutron wavelength of λ=6 Å and a detector offset of 25 cm with two sample-detector distances of 1.33 and 7.0 m were utilized to achieve a Q range of 0.0048-0.46 Å⁻¹. The net intensities were corrected for the background and empty cell (pure D₂O), accounting for the detector efficiency using the scattering from an isotropic scatterer (Plexiglas), and converted to an absolute differential cross section per unit sample volume (in units of cm⁻¹) using an attenuated empty beam. The data were then corrected for incoherent scattering by subtracting a constant background. The shape-reconstruction algorithm GA_STUCT (11) was used to generate solution conformations, similar to previous studies (10, 11). Beginning with an initial guess of randomly-distributed scattering centers, the program rearranges the position of the scattering centers to best fit the experimental scattering data.

The weight-average molecular weight (M_{w}) of each sample was calculated from the equation

$${M}_{W} = \frac{1000 I ( 0 ) {N}_{A}}{c {{\overline{υ}}^{2} ( {ρ}_{P} - {ρ}_{S} )}^{2}} , \qquad ( 1 )$$

where ρS and ρP are the scattering length densities of the solvent (6.36×10¹⁰ cm⁻²) and protein (3.23×10¹⁰ cm⁻²), respectively, c is the protein concentration (11.6 mg/mL at pH 3 and 11.4 mg/mL at pH 7), and υ̅ is the protein specific volume (0.734 cm³/g). (25) I(0)-values were determined from Guinier plots (25) using I(Q)=I(0)exp(−Q²R_{g}²/3), where R_{g} is the radius of gyration. Guinier plots, generally valid for QR_{g}<1.3, can be influenced by solution structuring due to intermolecular interactions between charged proteins, which becomes increasingly important as Q decreases below 1.5/R (10) (or <0.05 Å⁻¹ using an α-Ch radius ˜30 Å). Thus, pair distance distribution functions were calculated from the SANS data using the program GNOM (26) according to the equation

$$I ( Q ) = 4 π {\int }_{0}^{{D}_{max}} P ( r ) \frac{sin ( Q r )}{Q r} d r , \qquad ( 2 )$$

where P(r) is related to the probability of two scattering centers (nuclei for SANS) being a distance r+dr apart, and Dₘₐₓ is the maximum distance between scattering centers within the protein or protein oligomer. I(0)-values were then obtained from the PDDFs through I(0)=4π∫₀^{D}^{ₘₐₓ}P(r)dr, which has the advantage of utilizing the entire Q-range to determine I(O), as opposed to just the low-Q values as in Guinier analysis (27).

The small-angle X-ray scattering data were measured using the X21 beamline at the National Synchrotron Light Source at the Brookhaven National Laboratory. (28) The X-ray wavelength was set to 1.24 Å with a pair of Si(111) monochromator crystals. The sample-to-detector distance was calibrated to be 1.69 m using a silver behenate standard. To avoid radiation damage, solutions were continuously passed at a flow rate of 60 μL/min through a 1-mm glass capillary housed within an aluminum block containing Plexiglas observation windows. (28) The net intensities were corrected for the background and solvent scattering as well as sample transmission, and were put on an absolute scale by comparison with a calibration standard (10 mg/mL BSA (10)).

Infrared spectra were measured with a Genesis II FT-IR spectrometer (Mattson Instruments). Solutions were loaded in a demountable liquid cell equipped with a circulating water jacket (T=20° C.) between a pair of CaF₂ windows using a 50 μm Teflon spacer. A liquid light guide (Oriel, model no. 77557) was used to directly illuminate the sample with UV or visible light for 2 hours prior to and during data collection, as previously described (9). The sample chamber was continuously purged with dry air to eliminate the influence of water vapor. For each spectrum, a 500-scan interferogram was collected with a 2 cm⁻¹ resolution. The relatively sharp surfactant peaks at ˜1600 cm⁻¹ were removed by subtracting the spectra measured for a pure surfactant solution under otherwise identical conditions, resulting in corrected spectra that were flat in the region between 2000 and 1750 cm⁻¹. Fourier self-deconvolution (FSD) was applied to spectra to resolve the overlapping bands in the Amide I region using a band-narrowing factor k=2.0 and a full width at half height of 12.6 cm⁻¹. Second derivative spectra were obtained with the Savitsky-Golay function for a 3^{rd} order polynomial, using a 13 data point window. Difference spectra were obtained by subtracting the spectra collecting under visible light from the spectra collecting under UV light illumination. Difference spectra obtained for pure α-chymotrypsin solutions without surfactant shown no significant absorbance changes (<1% throughout the amide I region).

Optical microscopy was performed on an Olympus IX71 inverted fluorescence microscope equipped using a 50× lens (Olympus, model SLCPlanFl) and a U-N41027 CAL CRIM C58158 filter cube (Chroma, model 058158). Images were recorded with a Hamamatsu digital CCD camera (model C4742-95). Aliquots (5-10 μL) of the protein-surfactant solution were deposited onto glass slides and dyed with an equal volume of a 400 μM Congo red aqueous solution.

Transmission electron microscopy was performed on a Philips EM420 TEM operating at 80 kV. A drop of protein solution was placed on a carbon-coated grid for 10 seconds and then blotted with filter paper, followed by repeating this procedure with a second drop. The grid was then placed in a freshly made 1 wt % uranyl acetate solution for 30 s.

### Results and Discussion

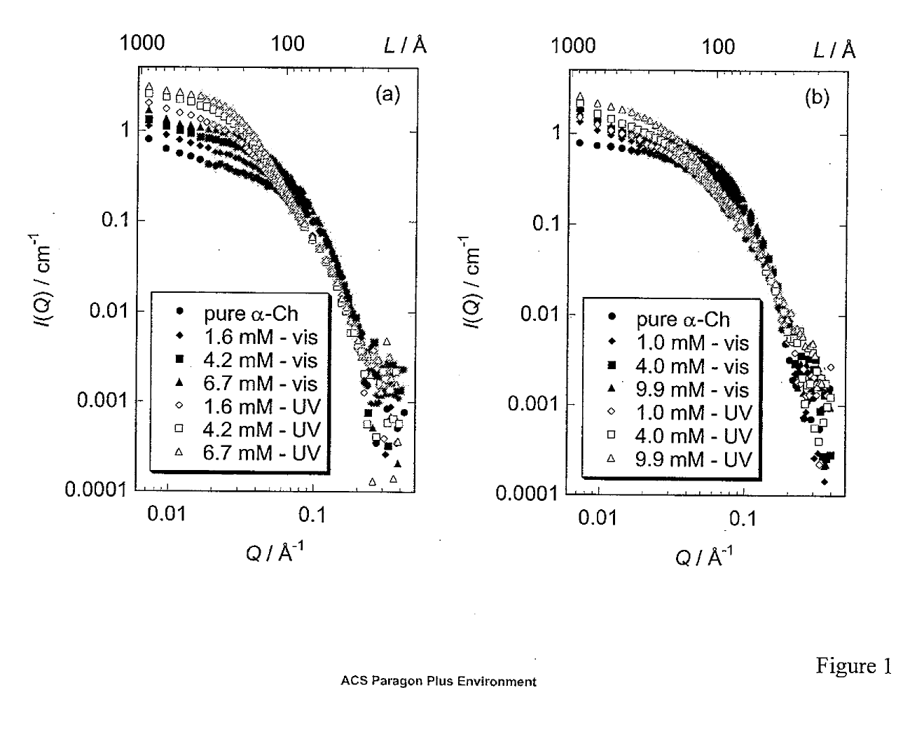

SANS data for α-Ch/azoTAB mixtures are shown in FIG. 1 as a function of pH, surfactant concentration, and light illumination. AzoTAB undergoes a photoisomerization to the relatively-hydrophilic cis form when illuminated with 350-nm UV light, which can be reversed back to the relatively-hydrophobic trans form upon exposure to 434-nm visible light. (20) In inverse space (e.g., with Q in units of Å⁻¹), the transitions responsible for SANS intensity changes in FIG. 1 can be difficult to conceptualize, thus, the real space length scale L (=2π/Q) is plotted on the upper x-axis. The addition of azoTAB causes an increase in scattering at low-Q (i.e., L>100 Å), suggesting the surfactant induces monomer→oligomer associations. UV-light illumination further enhances this effect, with a shift in the scattering curves to lower Q indicating greater protein aggregation when the surfactant is converted to the cis form. Thus, the trans isomer appears capable of replacing protein-protein interactions with protein-surfactant interactions. Beyond Q>0.2 Å⁻¹ (L<30 Å, or length scales less than the protein diameter) the SANS data converge, suggesting that the individual protein subunits remain relatively intact. However, at high Q the resolution (˜20 Å) of the SANS data is approached due to weak sample scattering relative to incoherent scattering from the protein (0.003 cm⁻¹) and solvent (0.0004 cm⁻¹ for 99.9% D₂O).

For associating systems, SANS has two advantageous properties. First, SANS is an absolute technique with the weight-average molecular weight (Me) of the sample given directly by I(0), the scattering at zero angle (see also Experimental Methods). Thus, the weight fraction of protein existing as monomer (x₁) and n-mer (xₙ=1−x₁) can be calculated from M_{w}=x₁M_{w,1}+(1−x₁)M_{w,n}, where M_{w,1} and M_{w,n} are the monomer and n-mer molecular weights, respectively. Second, SANS is additive with the scattering for a mixture of monomer (1-mer) and n-mer species given by the sum of the contributions from each oligomer o from 1 to n (29)

$$I ( Q ) = {n}_{p} ( {\sum }_{o = 1}^{n} \frac{{N}_{o}}{N} {\vert {F}_{o} ( Q ) \vert}^{2} ) \overline{S} ( Q ) ,$$

where

$${n}_{p} = \frac{1}{V} {\sum }_{o = 1}^{n} {N}_{o} = \frac{N}{V}$$

is the total number of particles per volume, Nₒ/N is the number fraction of a given type of oligomer, F₀(Q) is the form factor for that oligomer, and S̅(Q) is the averaged structure factor related to the partial structure factors Sᵢⱼ(Q). Hence for a non-interacting mixture of monomer and a single n-mer, (30) the scattering intensity can be shown to be I=ν₁I₁+νₙIₙ, where ν₁ and νₙ are the fractions of protein existing as monomer and n-mer on a volume basis (not to be confused with the volume fraction in solution, φ=c υ̅/1000, where c is the protein concentration in mg/mL units and υ̅ is the protein specific volume), while I₁ and Iₙ are the scattering from pure monomer and n-mer, respectively. (27) Since νᵢ˜xᵢ, assuming that υ̅ is constant independent of oligomeric state, the total scattering intensity is then also given by the linear combination I=x₁I₁+xₙIₙ. (31) Thus, these two properties of SANS can be utilized to assign the contributions of the overall scattering to the monomer and n-mer, followed by shape-reconstruction to determine the in vitro structure of α-Ch oligomers. In the sections that follow this will first be illustrated for pure α-Ch, then extended to solutions containing azoTAB to demonstrate photo-reversible α-Ch association.

### Pure Protein Solutions

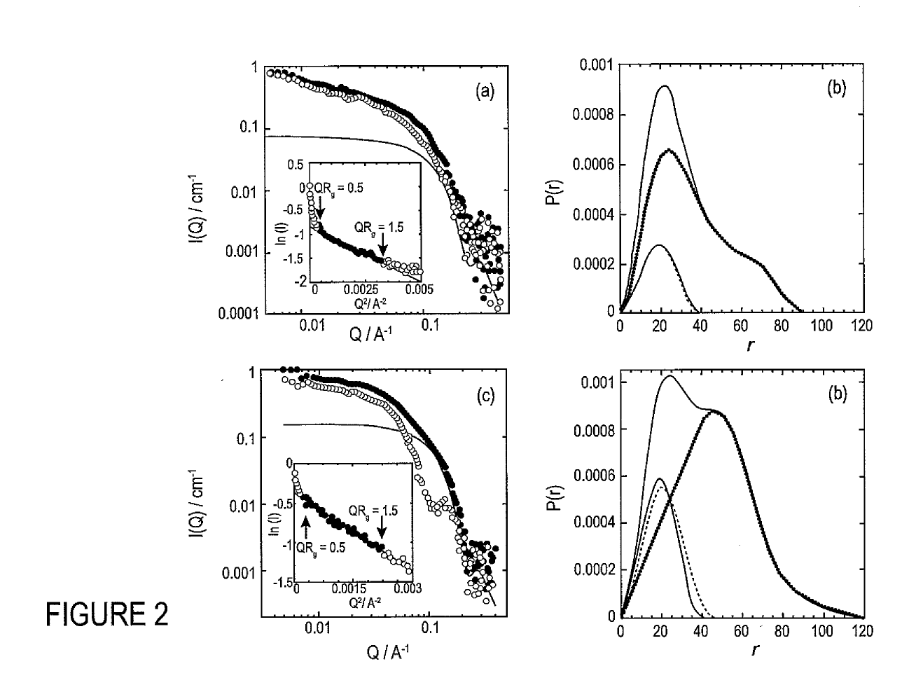

α-Ch is well-know to self associate through either a monomer-dimer (pH 3) or monomer-hexamer (pH 7) equilibrium at low ionic strength, (32-34) with a reduction in the overall positive charge of the protein (pI=9.1) with increased pH generally allowing for greater association. The SANS data for pure α-Ch solutions shown in FIG. 1, measured at conditions where self-association is expected to be prevalent (˜10 mg/mL protein), are re-plotted in FIG. 2. The raw data are largely featureless due to the presence of both monomer and oligomer in solution, complicating quantitative analysis of the self-association process. To deconvolute the scattering data, the weight-average molecular weight (M_{w}) of each sample was calculated from the scattering at zero angle, reported as the effective oligomer size (n^{eff}M_{w}/M₁) in Table 1. I(0) values determined from both Guinier plots (25) using I(Q)=I(0)exp(−Q²R_{g}²/3), technically valid for QR_{g}<1.3 as discussed below, where R_{g} is the radius of gyration (FIGS. 2a and 2c, insets), and from the entire Q-range using pair distance distribution functions (PDDFs) in FIGS. 2b and 2d, are generally consistent. Note that the steep upturn in the Guinier plots at Q<0.01 Å⁻¹ (L>600 Å) could be due to the presence of a small amount of higher-order aggregates, which due to the characteristic Q⁻⁴ decay would not be expected to influence the data analyses employed below.

From the M_{w}-value determined at each pH, the weight fraction of monomer and n-mer were calculated. The portion of the scattering resulting from free monomers, estimated from the monomer PDB file 2CHA using the program CRYSON (35) and scaled with a monomer concentration of x₁c as shown in FIGS. 2a and 2c, were then subtracted from the overall scattering to give the oligomer-only SANS data. This procedure presumes that the structure of the monomer in vitro is well represented by the native X-ray crystallographic structure, shown to be true for a range of soluble proteins (12, 36, 37). Compared to the featureless raw data, the oligomer scattering curve at pH 7 displays a prominent peak at Q=0.14 Å⁻¹, translating in real space to L ˜45 Å, also detected as a peak in the corresponding PDDF curve. This dimension corresponds to a highly probable distance within the protein oligomer, namely the separation distance between monomers. Peaks in this Q-range signify well-ordered oligomer conformations and are often used as qualitative tests for oligomer formation, (38) lending confidence in the deconvolution procedure. Oligomer peaks become more pronounced with increasing n-mer size, thus, it is not surprising that the dimer data at pH 3 do not display this peak.

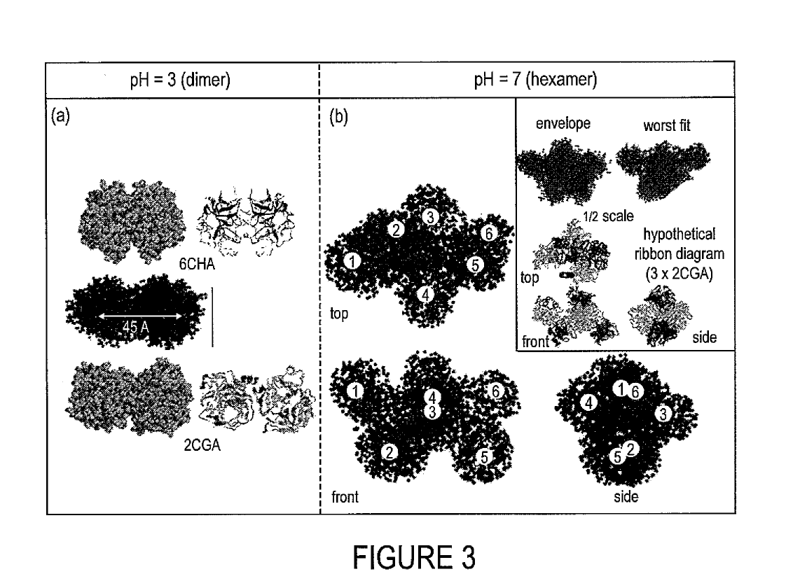

Following deconvolution, shape-reconstructions of the oligomer-only data were performed, conceptually similar to previous studies used to determine the in vitro structures of partially-folded BSA (10, 21) and lysozyme (11). The GA_STRUCT program begins with chains of randomly oriented “scattering centers” (i.e., atomic nuclei for SANS), with a genetic algorithm consisting of matings, mutations, and extinctions used to update the shape. (15) Despite this general procedure, the dimer (pH 3) and hexamer (pH 7) structures indeed contain n subunits for each n-mer, as shown in FIG. 3. Interestingly, the SANS-based in vitro dimer is not consistent with the “face-to-face” (active site-to-active site) crystal packing of α-chymotrypsin, and is instead better represented by the “back-to-side” packing of chymotrypsinogen (2CGA, note that α-Ch results from the removal of two dipeptides at positions 14-15 and 147-148 in chymotrypsinogen). For example, the maximum dimension of 6CHA ˜70 Å, while the PDDF in FIG. 2b gives a Dₘₐₓ of 90 Å compared to ˜85 Å for 2CGA. This serves to highlight the influence that crystal-packing constraints can have on molecular orientations, a significant advantage of SANS in the study of protein aggregates, and could explain why the role of the active site in α-Ch association remains unsolved in the literature with different techniques yielding conflicting results. (32)

Shape-reconstruction of the pH 7 data reveals the compact, “W-shaped” hexamer shown in FIG. 3b. The average distance between nearest-neighbor subunits is 43±5 Å, in agreement with the 0.14 Å⁻¹ peak in FIG. 2c, while the orientation angle between three successive subunits is 70±10°. The consistency of these values suggests that specific intermolecular interactions are responsible for hexamer formation in solution, resulting in the twisted arrangement of the subunits. Interestingly, the ribbon diagram of a hypothetical hexamer constructed by continuing the relative orientation of the two macromolecules in 2CGA (with alternate proteins color-coded blue and green) exhibits a similar twisted orientation, with all but the final protein in nearly identical locations. In contrast, the face-to-face arrangement of 6CHA would not support higher-order association, as opposed to the “heterologous association” apparently observed in FIG. 3a. (31) Also shown in the inset of FIG. 3 is the consensus envelope obtained by docking and averaging ten independent fits of the GA_STRUCT program, along with the run that statistically produced the worst fit to the data. Both of these structures agree with the W-shaped hexamer conformation, demonstrating that the coupled deconvolution/shape-reconstruction technique can be applied to protein oligomers in solution. Non-native protein conformations such as partially-folded or associated states challenge existing crystallographic and NMR methods. However, as demonstrated in FIG. 3 and in recent studies of photo-controlled protein folding (10, 11), SANS can provide valuable information on these important yet understudied class of structures.

α-chymotrypsin/azoTAB solutions

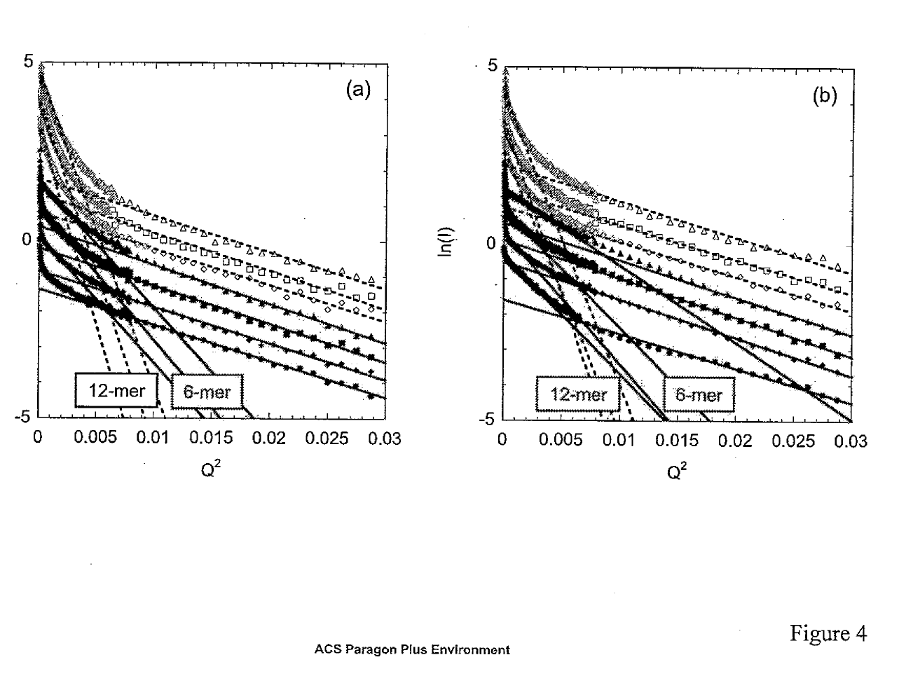

As discussed above, qualitative assessment of the SANS data in FIG. 1 indicate enhanced α-Ch association with either increased surfactant concentration or upon converting azoTAB from the trans to the cis form with UV-light illumination. To quantitatively investigate this phenomena, Guinier plots of the SANS data for α-Ch/azoTAB solutions were generated, as shown in FIGS. 4a and 4b. Two unique slopes can be detected at each condition, the first in the region of Q²<0.002 Å⁻² giving the z-average radius of gyration of the mixture, and the second at Q²˜0.01-0.03 Å⁻² with R_{g}-values ranging from 17.0-18.2 Å, as shown in Table 1. These latter values are consistent with the R_{g} of monomeric α-Ch in the literature of 16.9 Å (39), thus, indicating a monomer/n-mer equilibrium, (40, 41) similar to the monomer-oligomer equilibrium observed during the early stages of fibril formation of Aβ proteins. (1, 8) The 7% increase in R_{g} with the addition of azoTAB in Table 1 indicates that a slight unfolding of the protein could be the cause of increased association, consistent with the general observation that partially-unfolded protein conformations can lead to amyloid fibril formation. (1) It should be pointed out, however, that the Guinier region is strictly valid only for QR_{g}<1.3, while the above fits span Q²=0.01-0.03 Å⁻² (QR_{g}=1.7-3). Replacing [3j₁(QR_{g})/QR_{g}]² with the approximate expression exp(−Q²R_{g}²/3), as suggested by Guinier, (25) results in deviations on the order of 10% over this Q-range.

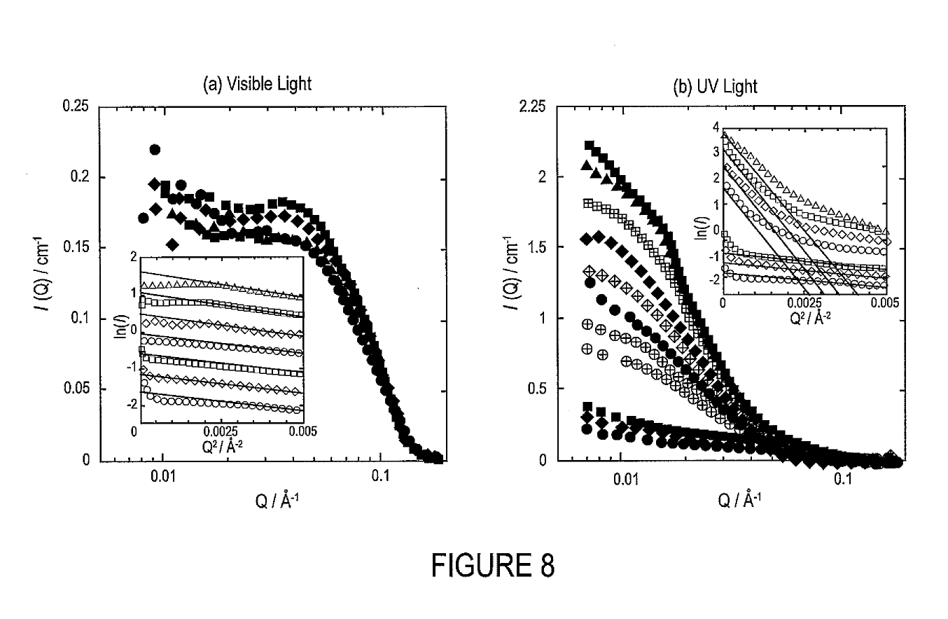

From the fits in the low-Q region of FIG. 4, the R_{g}-values are approximately constant at a given pH and light condition, suggesting that the oligomer size is primarily determined by the state of the surfactant. I (0)-values determined from either the Guinier plots in FIG. 4 for Q²<0.002 Å⁻² or PDDFs of the overall data (not shown) are displayed in Table 1, along with the effective oligomer size (nefo. Together, these values suggest monomer-hexamer equilibrium for the visible-light data and a monomer-dodecamer equilibrium under UV light, however, unlike pure α-chymotrypsin the oligomer size is not known a priori. Nevertheless, additional evidence will be presented below to support this type of protein self-association. In truth, these n-mer assignments are a result of a comprehensive iterative procedure whereby the number of protein subunits observed from shape reconstruction of the raw data (monomer+oligomer) were used to provide initial estimates of n. However, since fitting the overall data gives z-averaged shape of the protein, (42) which is heavily weighted towards the oligomer conformation, six and 12 subunits could be consistently detected even from the raw data (see below). Furthermore, for the UV-data, the choice of n=12 was particularly clear given that both the SANS and SAXS data (FIG. 8) appear to converge to an n^{eff} value of 12 with increasing surfactant concentration.

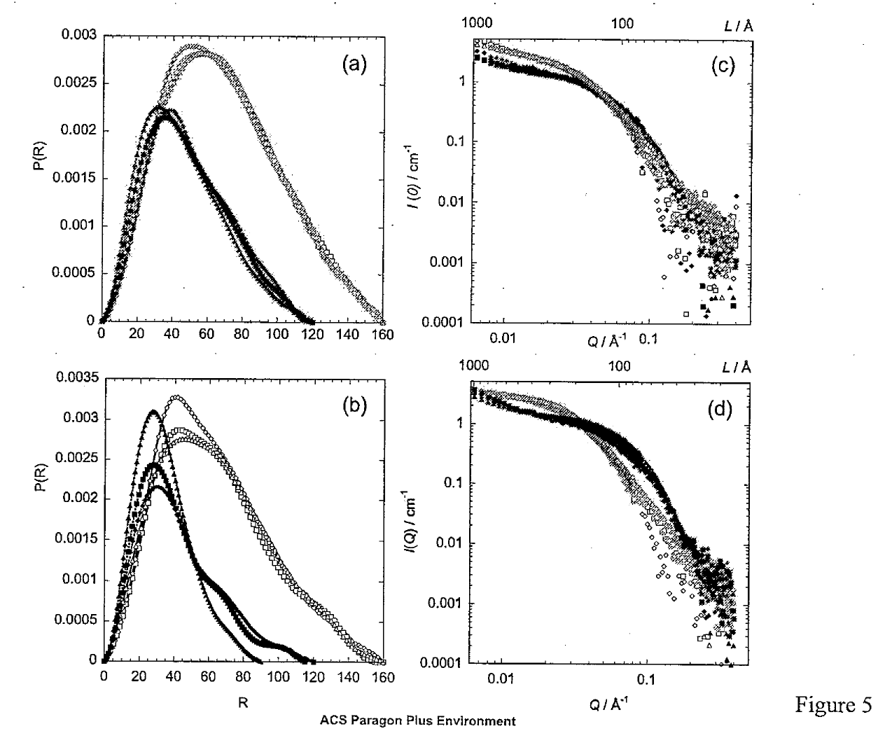

From these resulting n-mer assignments, the monomer weight fraction was calculated, indicating that the monomer-oligomer equilibrium shifts towards n-mer formation with increasing surfactant concentration, a likely result of increased partial unfolding as mentioned above. To gain further insight in the oligomer structures, the SANS data were deconvoluted as above to obtain the portion of the scattering due only to the n-mers. As shown in FIG. 5, the xₙ-scaled oligomer scattering data are largely consistent for a given pH and light conditions, suggesting a sound deconvolution procedure. Some subtle changes are observed with increasing surfactant concentration within a given data set, particularly in the high-Q region representing fine structural detail. Specifically, the peak observed at Q˜0.2 Å⁻², similar to the deconvoluted-hexamer of pure α-Ch at pH 7, becomes “washed out” with increasing azoTAB concentration, suggesting that the oligomers become more disordered with increased fluctuations in the protein subunit positions. Using Guinier plots to calculate of the radius of gyration from each oligomer-only scattering profile give the values of R_{g}ⁿ reported in Table 1.

PDDFs calculated from the oligomer-only data display a similar degree of homogeneity at each condition with increasing surfactant, as shown in FIGS. 5a and 5b. Interestingly, independent of oligomer type (hexamer or dodecamer) R_{g}ⁿ∝(M_{w})^{0.42±0.03} compared to the monomer radius of gyration, where M^{w} is the molecular weight of the oligomer. A similar scaling exponent (0.45) has been reported for self-associating insulin, with values intermediate between those expected for spheres (⅓) or Gaussian coils (½) suggesting relatively open oligomer structures. (30)

Comparing the visible light (hexamer) PDDFs to FIG. 2d for the pure hexamer reveals a shift in the PDDF peak to lower r-values. This suggests a potential unraveling of the tightly-packed W-shaped hexamer with the most probable dimension being reduced to distances within the protein subunits (e.g., the protein radius) as opposed to distances between the subunits. Although note that Dₘₐₓ of the hexamer remains at ˜120 Å as in FIG. 2d, thus, only partial unraveling can be occurring, largely retaining the twisted hexamer conformation. For example, a linear n-mer formed from a protein with a radius of 20 Å would give peaks at 20, 40, . . . , (n−1)40 Å. For the dodecamer structures the most probable dimension returns to 40-50 Å, while Dₘₐₓ undergoes a modest increase to ˜160 Å, hence, longitudinal extension of hexamers to form dodecamers does not appear to be an appropriate mechanism. Shoulders can also be detected in the PDDF curves at ˜80, 100, and 120 Å correspond to distances between higher-order neighbors, suggesting regular, as opposed to random, oligomer conformations. Guinier analyses of the oligomer-only data (not shown) give radii of gyration of the n-mers (R_{g}ⁿ) consistent with the PDDF analysis as displayed in Table 1, again largely independent of surfactant concentration across a given data set. Taken together, this evidence suggests that converting azoTAB to the cis form with UV light causes hexamers to laterally (as opposed to longitudinally) associate into dodecamers.

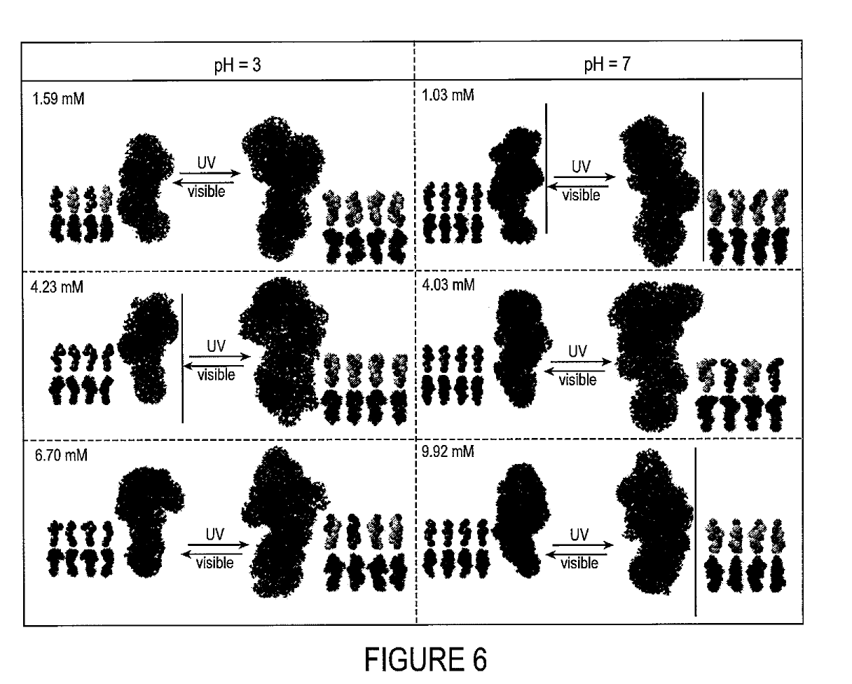

To obtain a better understanding of the oligomer conformations, shape-reconstruction was applied to the deconvoluted SANS data, as shown in FIG. 6. In all cases the shape-reconstruction algorithm returned conformations containing either six or 12 subdomains, despite the fact that the program begins with a random arrangement of scattering centers. This fact further confirms the choice of hexamers and dodecamers, as well as the overall deconvolution procedure. The shape-reconstructed hexamers indeed support the notion above of an unraveling of the W-shaped hexamer, as the hexamers now have extended, corkscrew-like appearances. Upon UV illumination and conversion of the surfactant to the cis form, hexamers are converted into the rope-like dodecamers, suggesting that dodecamer formation result from lateral association of two hexamers. This is illustrated by the bead-model structures accompanying each 90°-rotation view of the oligomers, used to guide the eye as to the relative positions of each protein subunit. The observed n-mer structures are found to be reasonably consistent across the range of pH and surfactant concentration conditions, again pointing to the global consistency of the deconvolution procedure.

### Photo-Induced α-Chymotrypsin Oligomers are Amyloid Precursors

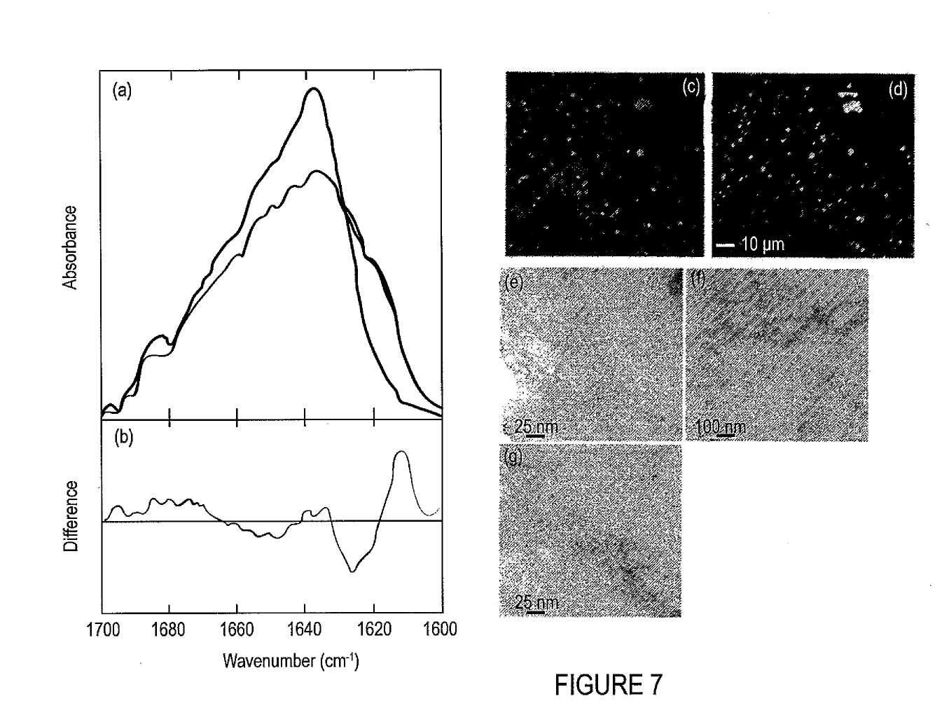

The lateral association of hexamers into dodecamers is consistent with the eventual rope-like conformation commonly observed in many amyloid fibrils, indicating that SANS may be reporting on the mechanism of formation of key prefibrillar intermediates in the amyloid cascade. To investigate whether the oligomer structures in FIG. 6 are true prefibrillar intermediates, several classic amyloid tests were performed on azoTAB/α-Ch mixtures. FT-IR spectra of pure α-Ch and α-Ch in the presence of azoTAB under both visible and UV light are shown in FIGS. 7a and 7b. Two aggregation processes can be triggered in the α-Ch/azoTAB system: the first upon the addition of trans azoTAB to pure α-Ch (dimers→hexamers at pH 3), and the second upon exposure of the α-Ch/azoTAB system to UV light (hexamers→dodecamers). As seen in the FT-IR spectra, both of these association processes give rise to an increase in peaks at 1612 and 1685 cm⁻¹, characteristic of intermolecular β-sheet formation, (5, 43, 44) at the expense of the peak at 1637 cm⁻¹ commonly assigned to intramolecular β-sheets. (45, 46) Zurdo et al. observe bands at 1612 and 1985 cm⁻¹ in SH3-domain protofibril intermediates that eventually mature into fully-developed amyloid fibrils, (44) suggesting that the oligomers observed in FIG. 6 are indeed precursors to amyloid structures.

The photomicrographs shown in FIGS. 7c and 7d further support this conclusion. Congo red staining of a α-Ch/azoTAB solution aged for five days results in characteristic Congo red fluorescence as well as “apple green” birefringence, respectively. Congo red preferentially stains amyloid structures due to the planar structure of the dye favoring incorporation into the β-sheet structure of amyloids. (47-49) These images were also accompanied by Maltese-cross patterns under cross polarizers (not shown) indicative of spherulites formed by the aligning of fibrils in a radial pattern. (50)

TEM images in FIG. 7 further demonstrate the formation of fibrillar structures. FIGS. 7e and 7f were obtained two weeks after preparing a fresh α-Ch/azoTAB solution, while FIG. 7g was obtained from an original SANS solution (pH 3, [azoTAB]=4.2 mM) approximately one year after collecting the SANS spectra. The fibrils shown in FIGS. 7e-g possess clear amyloid characteristics: they are long, unbranched, and appear to be twisted, with diameters of ca. 10 nm. Combined, these tests confirm that the samples used in SANS are indeed pre-amyloid oligomer intermediates.

### Photo-Reversible Protein Association

To investigate photoreversible protein association, small-angle X-ray scattering (SAXS) data were collected for mixtures of chymotrypsinogen-A and azoTAB at pH 3, as shown in FIG. 8. Chymotrypsinogen is the zymogen of α-chymotrypsin, activated by the removal of two dipeptides at positions Ser14-ArglS and Thr147-Asn148 leading to the formation of the active site. (51) Despite this structural similarity, however, chymotrypsinogen does not generally associate in solution unlike in the case of α-chymotrypsin. (52, 53)

This phenomena is supported by the visible-light SAXS data in FIG. 8a, where a clear intermolecular interaction peak is observed in contrast to FIG. 1, consistent with increasing electrostatic repulsion between chymotrypsinogen monomers as the cationic surfactant binds to the positively-charged protein. A Guinier plot of the pure-protein SAXS data (FIG. 8 inset) gives R_{g}=17.1 Å, similar to the SAXS-derived R_{g}-value from the literature of 17.6 Å. (39) With increasing surfactant concentration, R_{g} increases modestly up to 10 mM azoTAB under visible light, eventually increasing to 19.7 Å at 24 mM azoTAB (Table 2). The enhanced negative deviations from the Guinier behavior at low Q with increasing surfactant concentration are a result of increasing intermolecular interactions.

Under UV light illumination, however, the situation is markedly different, with large increases in the SAXS data observed at low Q (note that the y-axes of FIGS. 8a and 8b differ by an order of magnitude), particularly at 10 mM azoTAB and beyond, coincidentally the surfactant concentration where the onset of chymotrypsinogen unfolding was observed under visible light. The Guinier plots under UV light also reveal the development of an additional larger species with increasing surfactant concentration, detected by the appearance of a steep slope at low Q. I(0) values for samples under visible light (˜0.2-0.25 cm⁻¹, see Table 2, where the SAXS data have been put on absolute scale by comparing to a calibration standard of 10 mg/mL BSA (10)) are consistent with the value expected for the monomer (I(0)=0.24 cm⁻¹), again indicating that chymotrypsinogen association does not occur under visible light. Under UV light a 12-fold increase in I(0) is observed at 19 mM and 24 mM azoTAB relative to the monomer data, suggesting that the association equilibrium is pushed entirely towards dodecamers, providing independent confirmation of the α-chymotrypsin data.

Reversibility of protein self-association is shown in FIG. 8b, where SAXS spectra were collected for UV-equilibrated samples following re-exposure to 434-nm visible light. The low-Q scattering decreases as a function of visible-light exposure time, with apparently several hours required for complete visible-light induced dissociation (beyond the limit of the allocated SAXS beam time). However, it should be pointed out that this dissociation process is not limited by the cis→trans isomerization kinetics, which occurs within minutes (20). Protein association and dissociation can generally occur on time scales ranging from seconds up to hours or even several days. (54, 55) Thus, the SAXS data demonstrate photoreversible control of protein oligomerization.

Obviously, many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

The following references are cited herein. The entire disclosure of each reference is relied upon and incorporated by reference herein.
1. Chiti, F., and Dobson, C. M. (2006) Protein misfolding, functional amyloid, and human disease, Annu., Rev. Biochem. 75, 333-366.
2. Sambashivan, S., Liu, Y., Sawaya, M. R., Gingery, M., and Eisenberg, D. (2005) Amyloid-like fibrils of ribonuclease A with three-dimensional domain-swapped and native-like structure, Nature (London) 437, 266-269.
3. Booth, D. R., Sunde, M., Bellotti, V., Robinson, C. V., Hutchinson, W. L., Fraser, P. E., Hawkins, P. N., Dobson, C. M., Radford, S. E., Blake, C. C. F., and Pepys, M. B. (1997) Instability, unfolding and aggregation of human lysozyme variants underlying amyloid fibrillogenesis, Nature 385, 787-793.
4. Brange, J., Andersen, L., Laursen, E. D., Meyn, G., and Rasmussen, E. (1997) Toward Understanding Insulin Fibrillation, J. Pharm. Sci. 86, 517-525.
5. Pallares, I., Vendrell, J., Aviles, F. X., and Ventura, S. (2004) Amyloid Fibril Formation by a Partially Structured Intermediate State of α-Chymotrypsin, J. Mol. Biol. 342, 321-331.
6. Klein, W. L., Stine, W. B., and Teplow, D. B. (2004) Small assemblies of unmodified amyloid b-protein are the proximate neurotoxin in Alzheimer's disease, Neurobiol. Aging 25, 569-580.
7. Kirkitadze, M. D., Bitan, G., and Teplow, D. B. (2002) Paradigm shifts in Alzheimer's disease and other neurodegenerative disorders: the emerging role of oligomeric assemblies, J. Neurosci. Res. 69, 567-577.
8. Yong, W., Lomakin, A., Kirkitadze, M. D., Teplow, D. B., Chen, S.-H., and Benedek, G. B. (2002) Structure determination of micelle-like intermediates in amyloid b-protein fibril assembly by using small angle neutron scattering, Proc. Nat. Acad. Sci. USA 99, 150-154.
9. Wang, S., and Lee, J., C. Ted. (2006) Protein Secondary Structure Controlled with Light and Photoresponsive Surfactants, J. Phys. Chem. B 110, 16117-16123.
10. Lee, C. T., Jr., Smith, K. A., and Hatton, T. A. (2005) Photocontrol of Protein Folding: The Interaction of Photosensitive Surfactants with Bovine Serum Albumin, Biochemistry 44, 524-536.
11. Hamill, A. C., Wang, S.-C., and Lee, C. T., Jr. (2005) Probing lysozyme conformation with light reveals a new folding intermediate, Biochemistry 44, 15139-15149.
12. Timchenko, A. A., Ptitsyn, O. B., Dolgikh, D. A., and Fedorov, B. A. (1978) The structure of ribonuclease in solution does not differ from its crystalline structure, FEBS Lett. 88, 105-8.
13. Chen, S. H., and Teixeira, J. (1986) Structure and fractal dimension of protein-detergent complexes, Phys. Rev. Lett. 57, 2583-6.
14. Svergun, D. I., Petoukhov, M. V., and Koch, M. H. J. (2001) Determination of domain structure of proteins from x-ray solution scattering, Biophys. J. 80, 2946-2953.
15. Heller, W. T., Krueger, J. K., and Trewhella, J. (2003) Further Insights into Calmodulin-Myosin Light Chain Kinase Interaction from Solution Scattering and Shape Restoration, Biochemistry 42, 10579-10588.
16. Marone, P. A., Thiyagarajan, P., Wagner, A. M., and Tiede, D. M. (1998) The association state of a detergent-solubilized membrane protein measured during crystal nucleation and growth by small-angle neutron scattering, J. Cryst. Growth 191, 811-819.
17. Hunt, J. F., McCrea, P. D., Zaccai, G., and Engelman, D. M. (1997) Assessment of the aggregation state of integral membrane proteins in reconstituted phospholipid vesicles using small angle neutron scattering, J. Mol. Biol. 273, 1004-1019.
18. Chiti, F., Taddei, N., Bucciantini, M., White, P., Ramponi, G., and Dobson, C. M. (2000) Mutational analysis of the propensity for amyloid formation by a globular protein, EMBO J. 19, 1441-1449.
19. Srisailam, S., Kumar Thallampuranam Krishnaswamy, S., Rajalingam, D., Kathir Karuppanan, M., Sheu, H.-S., Jan, F.-J., Chao, P.-C., and Yu, C. (2003) Amyloid-like fibril formation in an all beta-barrel protein. Partially structured intermediate state(s) is a precursor for fibril formation, J. biol. chem. 278, 17701-9.
20. Le Ny, A.-L. M., and Lee, C. T., Jr. (2006) Photoreversible DNA Condensation Using Light-Responsive Surfactants, J. Am. Chem. Soc. 128, 6400-6408.
21. Wang, S. S. S., Chen, Y.-T., and Chou, S.-W. (2005) Inhibition of amyloid fibril formation of b-amyloid peptides via the amphiphilic surfactants, Biochimica et BBA—Mol. Basis. Dis. 1741, 307-313.
22. Hayashita, T., Kurosawas, T., Miyata, T., Tanaka, K., and Igawa, M. (1994) Effect of structural variation within cationic azo-surfactant upon photoresponsive function in aqueous solution, Colloid Polym. Sci. 272, 1611-19.
23. Shang, T., Smith, K. A., and Hatton, T. A. (2003) Photoresponsive Surfactants Exhibiting Unusually Large, Reversible Surface Tension Changes under Varying Illumination Conditions, Langmuir 19, 10764-10773.
24. Glinka, C. J., Barker, J. G., Hammouda, B., Krueger, S., Moyer, J. J., and Orts, W. J. (1998) The 30 m small-angle neutron scattering instruments at the National Institute of Standards and Technology, J. Appl. Cryst. 31, 430-445.
25. Guinier, A. a. F., G. (1955) Small Angle Scattering of X-ray, Wiley, New York.
26. Svergun, D. I. (1992) Determination of the Regnlarization Parameter in Indirect-Transform Methods Using Perceptual Criteria, J. Appl. Cryst. 25, 495-503.
27. Svergun, D. I., and Koch, M. H. J. (2003) Small-angle scattering studies of biological macromolecules in solution, Reports on Progress in Physics 66, 1735-1782.
28. Yang, L. (2005) The X21 SAXS instrument at NSLS for studying macromolecular systems, Macromol. Res. 13, 538-541.
29. Chen, S.-H., and Lin, T.-L. (1987) in Neutron Scattering (Price, D. L., and Sköld, K., Eds.) pp 489-543, Academic Press, San Diego.
30. Pedersen, J. S., Hansen, S., and Bauer, R. (1994) The aggregation behavior of zinc-free insulin studied by small-angle neutron scattering, Eur. Biophys. J. 22, 379-89.
31. Shilton, B. H., McDowell, J. H., Smith, W. C., and Hargrave, P. A. (2002) The solution structure and activation of visual arrestin studied by small-angle X-ray scattering, Eur. J. Biochem. 269, 3801-3809.
32. Ramakrishna, T., and Pandit, M. W. (1992) Involvement of active site in self-association of proteins: a case of alpha-chymotrypsin, Indian J. Biochem. Biophys. 29, 192-200.
33. Pandit, M. W., and Rao, M. S. N. (1975) Self-association of proteins. Self-association of a-chymotrypsin at its isoelectric point in buffer solutions of ionic strength 0.1, Biochemistry 14, 4106-10.
34. Ackers, G. K., and Thompson, T. E. (1965) Determination of stoichiometry and equilibrium constants for reversibly associating systems by molecular sieve chromatography, P. Nat. Acad. Sci. USA 53, 342-9.
35. Svergun, D. I., Richard, S., Koch, M. H. J., Sayers, Z., Kuprin, S., and Zaccai, G. (1998) Protein hydration in solution: experimental observation by x-ray and neutron scattering, P. Nat. Acad. Sci. USA. 95, 2267-2272.
36. Durchschlag, H., and Zipper, P. (1996) Comparative determination of structural parameters and conformational changes of proteins by small-angle scattering, crystallography and hydrodynamic analysis, J. Mol. Struct. 383, 223-229.
37. Ninio, J., Luzzati, V., and Yaniv, M. (1972) Comparative small-angle x-ray scattering studies on unacylated, acylated, and crosslinked Escherichia coli transfer RNAIVa1, J. Mol. Biol. 71, 217-29.
38. Berret, J.-F., and Oberdisse, J. (2004) Electrostatic self-assembly in polyelectrolyte-neutral block copolymers and oppositely charged surfactant solutions, Physica B 350, 204-206.
39. Perkins, S. J., Smith, K. F., Kilpatrick, J. M., Volanakis, J. E., and Sim, R. B. (1993) Modelling of the serine-proteinase fold by X-ray and neutron scattering and sedimentation analyses: occurrence of the fold in factor D of the complement system, Biochem. J. 295 (Pt 1), 87-99.
40. Fujisawa, T., Ueki, T., Inoko, Y., and Kataoka, M. (1987) X-ray scattering from a troponin C solution and its interpretation with a dumbbell-shaped-molecule model, J. Appl. Cryst. 20, 349-55.
41. Tranier, S., Mortier-Barriere, I., Ilbert, M., Birck, C., Iobbi-Nivol, C., Mejean, V., and Samama, J.-P. (2002) Characterization and multiple molecular forms of TorD from Shewanella massilia, the putative chaperone of the molybdoenzyme TorA, Protein Sci. 11, 2148-2157.
42. Vanhoudt, J., Abgar, S., Aerts, T., and Clauwaert, J. (2000) A small-angle X-ray solution scattering study of bovine a-crystallin, Eur. J. Biochem. 267, 3848-3858.
43. Jaikaran, E. T. A. S., Higham, C. E., Serpell, L. C., Zurdo, J., Gross, M., Clark, A., and Fraser, P. E. (2001) Identification of a Novel Human Islet Amyloid Polypeptide b-Sheet Domain and Factors Influencing Fibrillogenesis, J. Mol. Biol. 308, 515-525.
44. Zurdo, J., Guijarro, J. I., Jimenez, J. L., Saibil, H. R., and Dobson, C. M. (2001) Dependence on Solution Conditions of Aggregation and Amyloid Formation by an SH3 Domain, J. Mol. Biol. 311, 325-340.
45. Susi, H., and Byler, D. M. (1986) Resolution-enhanced Fourier transform infrared spectroscopy of enzymes, Method. Enzymol. 130, 290-311.
46. Byler, D. M., and Susi, H. (1986) Examination of the secondary structure of proteins by deconvolved FTIR spectra, Biopolymers 25, 469-87.
47. Klunk, W. E., Jacob, R. F., and Mason, R. P. (1999) Quantifying amyloid by Congo red spectral shift assay, Method. Enzymol. 309, 285-305.
48. Glenner, G. G. (1980) Amyloid deposits and amyloidosis. I. The b-fibrilloses, New Engl. J. Med. 302, 1283-92.
49. Puchtler, H., and Sweat, F. (1965) Congo red as a stain for fluorescence microscopy of amyloid, J. Histochem. Cytochem. 13, 693-4.
50. Krebs, M. R. H., Bromley, E. H. C., Rogers, S. S., and Donald, A. M. (2005) The mechanism of amyloid spherulite formation by bovine insulin, Biophys. J. 88, 2013-2021.
51. Wang, D., Bode, W., and Huber, R. (1985) Bovine chymotrypsinogen A. X-ray crystal structure analysis and refinement of a new crystal form at 1.8 ANG. resolution, J. Mol. Biol. 185, 595-624.
52. Rao, M. S. N., and Kegeles, G. (1958) An ultracentrifuge study of the polymerization of a-chymotrypsin, J. Am. Chem. Soc. 80, 5724-9.
53. Egan, R., Michel, H. O., Schlueter, R., and Jandorf, B. J. (1957) Physicochemical investigation of the chymotrypsins. II. On the mechanism of dimerization of chymotrypsin, Arch. Biochem. Biophys. 66, 366-73.
54. Ding, F., and Dokholyan, N. V. (2005) Simple but predictive protein models, Trends Biotechnol. 23, 450-455.
55. Dokholyan, N. V. (2006) Studies of folding and misfolding using simplified models, Curr. Opin. Struc. Biol. 16, 79-85.

What is claimed is:

1. A method for determining the structure of partially-folded proteins in non-native conformations and supramolecular complexes undergoing self- or hetero-association in solution comprising:
a) allowing proteins to interact with photosensitive surfactants containing an azobenzene group;
b) exposing said proteins and surfactants to light illumination;
c) determining the small-angle neutron scattering (SANS) of said proteins; and
d) applying SANS data to shape-reconstruction analysis,
wherein said surfactant undergoes photoisomerization upon exposure to light.

2. The method according to claim 1, wherein said protein is an amyloid-forming protein.

3. The method according to claim 1, wherein said protein is 6-chymotrypsin.

4. The method according to claim 1, wherein said surfactant is azobenzene-trimethylammonium bromide.

5. The method according to claim 1, wherein said non-native conformations are prefibrillar intermediates.

6. The method according to claim 5, wherein said prefibrillar intermediate is a protofibril, a protofilament, or a fibril intermediate.

7. The method according to claim 1, wherein said light is visible or UV light.

8. A method of using light illumination to induce photoreversible changes in both the secondary and tertiary structure of proteins comprising:
a) allowing said proteins to interact with photosensitive surfactants containing an azobenzene group; and
b) exposing said proteins and said surfactants to light illumination, wherein said surfactant undergoes photoisomerization upon exposure to light and wherein the isomerization reverses when the light exposure is removed.

9. The method according to claim 8, wherein said protein is an amyloid-forming protein.

10. The method according to claim 8, wherein said protein is \acute{α}-chymotrypsin.

11. The method according to claim 8, wherein said surfactant is azobenzene-trimethylammonium bromide.

12. The method according to claim 8, wherein said non-native conformations are prefibrillar intermediates.

13. The method according to claim 12, wherein said prefibrillar intermediate is a protofibril, a protofilament, or a fibril intermediate.

14. The method according to claim 8, wherein said light is visible or UV light.

15. A method of generating conformations of partially-folded proteins in non-native conformations in solution comprising:
a) determining small-angle neutron scattering (SANS) of said proteins intermediates; and
b) applying SANS data to the shape-reconstruction algorithm GA_STUCT,
wherein the weight-average molecular weight (M_{w}) is calculated from the equation
$${M}_{W} = \frac{1000 I ( 0 ) {N}_{A}}{c {{\overline{υ}}^{2} ( {ρ}_{P} - {ρ}_{S} )}^{2}} ,$$
where ρS and ρP are the scattering length densities of the solvent (6.36×10¹⁰ cm⁻²) and protein (3.23×10¹⁰ cm⁻²), respectively, c is the protein concentration (11.6 mg/mL at pH 3 and 11.4 mg/mL at pH 7), and υ̅ is the protein specific volume (0.734 cm³/g,
I(0)-values were determined from Guinier plots using
I(Q)=I(0)exp(−Q²R_{g}²/3), where R_{g} is the radius of gyration,
pair distance distribution functions were calculated from the SANS data according to the equation
$$I ( Q ) = 4 π {\int }_{0}^{{D}_{max}} P ( r ) \frac{sin ( Q r )}{Q r} d r ,$$
where P(r) is related to the probability of two scattering centers (nuclei for SANS) being a distance r+dr apart, and Dₘₐₓ is the maximum distance between scattering centers within the protein or protein oligomer, and I(0)-values were then obtained from the PDDFs through I(0)=4π∫₀^{D}^{ₘₐₓ}P(r)dr.
